(19) United States
(12) Patent Application Publication
(10) Pub. No.: US 2013/0102078 A1
(43) Pub. Date: Apr. 25, 2013

(54) NANOINJECTION SYSTEM DNA PLACEMENT PIPETTE COUPLER

(71) Applicant: NanoInjection Technologies, L.L.C., Salt Lake City, UT (US)

(72) Inventor: Quentin T. Aten, Orem, UT (US)

(73) Assignee: NANOINJECTION TECHNOLOGIES, L.L.C., Salt Lake City, UT (US)

(21) Appl. No.: 13/657,689

(22) Filed: Oct. 22, 2012

Related U.S. Application Data
(60) Provisional application No. 61/550,190, filed on Oct. 21, 2011.
(60) Provisional application No. 61/550,202, filed on Oct. 21, 2011.

(51) Int. Cl. C12M 1/00; B65B 3/04; C12N 15/85

(52) U.S. Cl. 435/455; 435/285.1; 141/2

(57) ABSTRACT

Systems, devices, and methods for injecting biological material into a micro-object such as a cell are provided. In one aspect, for example, a cellular injection device can include a housing, an injection lance coupled to the housing and having a working tip extending outward from the housing, and a biological material delivery device coupled to the housing and having an effluent tip extending outward from the housing. The effluent tip can be positioned sufficiently proximal to the working tip such that biological material expelled from the effluent tip substantially contacts the working tip. In one aspect, the injection lance is removably coupled to the housing. In another aspect, the biological material delivery device is removably coupled to the housing.

### PRIORITY DATA

This application claims the benefit of U.S. Provisional Patent application Ser. No. 61/550,190, filed on Oct. 21, 2011, and U.S. Provisional Patent Application Ser. No. 61/550,202, filed on Oct. 21, 2011, both of which are incorporated herein by reference.

### BACKGROUND OF THE INVENTION

Microinjection of foreign materials into a biological structure such as a living cell can be problematic. Various transfection techniques include the microinjection of foreign genetic material such as DNA into the nucleus of a cell to facilitate the expression of foreign DNA. For example, when a fertilized oocyte (egg) is transfected, cells arising from that oocyte will carry the foreign genetic material. Thus in one application, organisms can be produced that exhibit additional, enhanced, or repressed genetic traits. In some cases, researchers have used microinjections to create strains of mice that carry a foreign genetic construct causing macrophages to auto-fluoresce and undergo cell death when exposed to a certain drugs. Such transgenic mice have since played roles in investigations of macrophage activity during immune responses and macrophage activity during tumor growth.

Prior art devices for restraining a cell or an embryo during micromanipulation generally consist of hollow capillary tubes with polished ends. In some cases suction is applied to the capillary to secure the embryo on the end of the capillary. In other cases, the embryo can be rotated by alternately applying suction and pressure while moving the capillary to expel and secure the embryo, now in a rotated orientation, at the tip of the capillary. Researchers have produced various mobile embryo restraints employing movable tweezer-like structures, or graspers with moveable finger-like elements. These mobile restraints have not found wide use in the manipulation of embryos.

### SUMMARY

The present disclosure provides devices, systems, and methods for injecting biological material into a micro-object such as a cell. In one aspect, for example, a cellular injection device can include a housing, an injection lance coupled to the housing and having a working tip extending outward from the housing, and a biological material delivery device coupled to the housing and having an effluent tip extending outward from the housing. The effluent tip can be positioned sufficiently proximal to the working tip such that biological material expelled from the effluent tip substantially contacts the working tip. In one aspect, the injection lance is removably coupled to the housing. In another aspect, the biological material delivery device is removably coupled to the housing.

In some example devices, a counter electrode can be coupled to the housing and electrically isolated from the injection lance. In one aspect, the counter electrode is positioned relative to the lance to complete an electrical circuit in proximity to the working tip during use in a liquid medium. In one specific aspect, the distance from the effluent tip to the working tip is from about 25 microns to about 500 microns. In another aspect, the distance from the effluent tip to the working tip is from about 100 microns to about 300 microns. In a further aspect, the proximity between the effluent tip and working tip is sufficient to allow the biological material to directly contact the working tip as the biological material is ejected from the effluent tip.

In some aspects, the housing can include electrical contacts that provide electrical connection from the lance to an electrical charging device. Additionally, in some aspects the biological material delivery device can further include fluidic tubing functionally coupled thereto, where the fluidic tubing is coupled to the housing to provide strain relief.

In some aspects, a cellular injection device support can be used to removably couple the cellular injection device to a micromanipulation device. In one aspect, the cellular injection device support provides electrical coupling between the injection lance and an electrical charging system.

In another aspect of the present disclosure, a method of electrostatically associating a biological material to a lance for subsequent delivery into a cell is provided. Such a method can include positioning an injection lance having a working tip in a liquid medium, charging at least the working tip of the lance with a charge having a polarity opposite the biological material, and positioning a biological material delivery device having an effluent tip in the liquid medium. The effluent tip is thus in proximity to the working tip. Subsequently, the biological material can be ejected from the effluent tip to the working tip to allow electrostatic association with the working tip. In one aspect, the working tip of the injection lance and the effluent tip of the biological material delivery device are fixed relative to one another by a housing prior to positioning in the liquid medium.

In another aspect, a method of injecting biological material into a cell can include introducing a cellular injection device into a liquid medium containing a cell, electrically charging the injection lance of the injection device with a charge having a polarity opposite the biological material, and ejecting the biological material from the effluent tip to the working tip to allow electrostatic association with the working tip. The injection lance can then be inserted into the cell, the injection lance can be discharged to release the biological material, and the lance can be withdrawn from the cell.

### DEFINITIONS OF TERMS

In describing and claiming the present invention, the following terminology will be used in accordance with the definitions set forth below.

The singular forms “a,” “an,” and, “the” can include plural referents unless the context clearly dictates otherwise. Thus, for example, reference to “a cell” can include reference to one or more of such cells, and reference to “the lance” can include reference to one or more of such lances.

As used herein, the term “micro-object” is used to describe objects of a size on a micro scale. One exemplary range for the term “micro-object” can be an object having an approximate diameter of from about 1 μm to about 1000 μm. Another range can be from about 10 μm to about 250 μm. It should be noted that the present scope contemplated object sizes of less than 1 μm, and that the present techniques can be utilized to restrain objects of any size capable of manipulation. The term “micro-object” can be used to describe both biological and non-biological material.

As used herein, the term “lance” refers to any structure or device that can be utilized to introduce biological material into a cell using electrostatic attraction. As such, “injection” as used herein can include any technique for introducing a biological material into a cell that involves a lance. It is also contemplated that a lance can be used to inject a biological material into a micro-object that may not necessarily be a cell.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint without affecting the desired result.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### BRIEF DESCRIPTION OF THE DRAWINGS

FIG. 1 is a graphical representation of an integrated injection device in accordance with one embodiment of the present disclosure.

FIG. 2 is a graphical representation of a cell and a portion of an integrated injection device in accordance with another embodiment of the present disclosure.

FIG. 3a is a graphical representation of an integrated injection device in accordance with another embodiment of the present disclosure.

FIG. 3b is a graphical representation of an integrated injection device in accordance with another embodiment of the present disclosure.

FIG. 3c is a graphical representation of an integrated injection device in accordance with another embodiment of the present disclosure.

FIG. 4a is a graphical representation of a coupling device in accordance with another embodiment of the present disclosure.

FIG. 4b is a graphical representation of a coupling device and an integrated injection device in accordance with another embodiment of the present disclosure.

FIG. 5 is a graphical depiction of steps to a method of electrostatically associating a biological material to a lance for subsequent delivery into a cell in accordance with another embodiment of the present disclosure.

### DETAILED DESCRIPTION

The present disclosure provides devices for delivering a biological material into a micro-object, as well as associated methods. In some aspects, such micro-objects can include biological objects such as individual cells, collections of cells, embryos, tissue, and the like. For example, in one aspect a cell can be injected with a biological material using such a device. It should be noted therefore, while the present disclosure refers often to cellular injection, the present devices, systems, and methods are not limited to use with micro-objects of a biological origin. For example, micro-beads, polymeric materials, and other micron-sized objects are also contemplated.

As one non-limiting example, DNA can be delivered into a cell or an organelle of a cell (e.g. a pronucleus), resulting in genomic integration of the DNA. Such delivery can be accomplished by a variety of techniques. One such technique involves the electrostatic association of a biological material with a delivery device, such as a lance, having an outer shape that is sufficiently small to deliver the biological material into the cell with reduced damage. A lance having a smaller outer shape may be less disruptive to cellular structures than traditional devices, and thus may allow delivery of the biological material into a cell with less cellular damage. As such, by charging the lance with an electrical charge that is opposite to the net charge of the biological material, the biological material will associate therewith when brought into contact with the lance. Once associated, the lance and the biological material can be inserted into the cell, and the lance can be discharged. The discharge of the lance causes the biological material to disassociate from the lance due to the decrease in the electrostatic force. As such, when the lance is removed from the cell, the biological material is retained therein.

One potential issue with this process relates to the association of the biological material with the lance. In many cases, a biological material in a liquid buffer will associate with an oppositely charged lance upon contact. Given the characteristics of soluble biological materials in a liquid, the molecules of the biological material readily diffuse throughout a liquid volume. As such, the probability of contact between a biological material and the lance decreases rapidly over time as diffusion occurs. The lance can be moved back and forth through the liquid to increase the probability of contact, however this can be highly inefficient. Thus, a large proportion of often costly biological materials can be lost in the liquid medium.

It is thus disclosed that various configurations and positions of device components of an injection system can facilitate a substantially higher proportion of biological material electrostatically associating with the lance, thus reducing waste and decreasing injection procedure time. In one aspect, for example, a biological material delivery device (e.g. a delivery pipette) can be brought in proximity to the lance tip. When the tips are in close proximity, the concentration of biological material in the ejection stream is high. By charging the lance and subsequently ejecting the biological material from the delivery pipette, biological material in the ejection stream has an increased probability of electrostatically accumulating at the tip. As such, the more accurate the positioning of the delivery pipette relative to the lance tip, the greater the likelihood of association for material in the ejection stream. While a variety of configurations are contemplated, it can be beneficial to position the delivery pipette such that all or substantially all of the biological material ejected therefrom passes in sufficiently close proximity to facilitate electrostatic association. In addition to proximity, the effluent tip of the delivery pipette can be designed and configured to further direct the ejection stream across the lance tip.

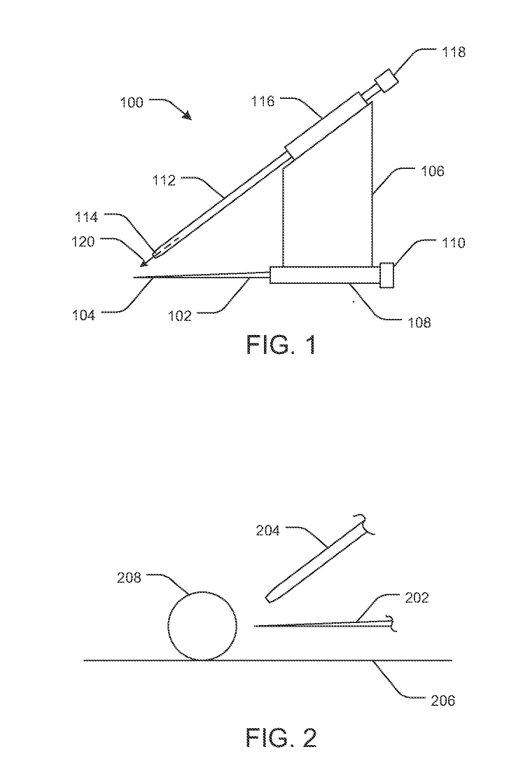

As is shown in FIG. 1, for example, an integrated injection device 100 can include a lance 102 having a working tip 104. The lance 102 can be coupled to a housing 106 by an attachment mechanism. The attachment mechanism can be any technique, device, or composition that can effectively couple the lance 102 to the housing 106. Thus it is contemplated that the lance 102 can be coupled to the housing 106 directly by, for example, adhesive bonding. In other aspects, the lance 102 can be coupled to the housing 106 using a lance mount 108, thus facilitating rapid replacement of the lance during use. A lance electrical contact 110 can be electrically coupled to the lance 102 to facilitate charging and discharging.

The integrated injection device 100 can also include a delivery pipette 112 having an effluent tip 114. The delivery pipette 112 can be coupled to the housing 106 by an attachment mechanism as was described for the lance 102. In one aspect, the delivery pipette 112 can be coupled to the housing 106 using a pipette mount 116. A fluidic coupling 118 can be associated with the delivery pipette 112 to allow coupling to a fluidic control device (not shown) capable of ejecting at least a portion of the biological material from the delivery pipette 112 in an ejection stream 120. As can be seen in FIG. 1, the effluent tip 114 is positioned relative to the working tip 104 to cause the ejection stream 120 to substantially contact the working tip 104. As such, the housing 106 maintains the relative positioning of the delivery pipette 112 relative to the lance 102 to facilitate such an interaction between the ejection stream 120 and the working tip 104.

The housing can be of any design and/or configuration that allow the delivery pipette and the lance to be maintained in a position or positions that facilitates a desired interaction between the tip of the lance and biological material ejected from the delivery pipette. Additionally, the materials used to construct the housing can vary widely depending on design and manufacturing preferences. In general, it may be useful to avoid electrical interaction between the housing material and the lance and/or the liquid medium. As such, in one aspect it can be beneficial to construct the housing from an electrically non-conductive material, such as a polymeric material. In other aspects, the housing material can be made from an electrically conductive material that is coated with a nonconductive film or layer.

In one aspect, it is contemplated that the housing can be disposable. In such cases, when the working life of either the lance or the delivery pipette is exhausted, the integrated injection device including the housing can be disposed of This may be upon wearing or damage to any of the components of the device, or it may be following a given number of injection procedures. In some aspects, it would be desirable to change the lance when a different biological material is to be used to avoid contamination. Thus, in such cases, the lance and/or the delivery pipette can be either permanently or temporarily affixed to the housing. In those aspects whereby the lance and/or the delivery pipette are to be disposed of and the housing reused, a temporary coupling would be beneficial. Such temporary coupling can be by any known technique that allows attachment and subsequent release of the components from the housing. Non-limiting examples can include interlocking mounts, screw connections, friction connections, temporary adhesives, luer connections, etc.

Furthermore, in some aspects the lance and/or the delivery pipette can be spatially adjusted relative to one another. For example, FIG. 2 shows a lance 202 and a delivery pipette 204 positioned relative to one another. The lance 202 is positioned above a support substrate 206 upon which rests a cell 208. The lance 202 can be positioned substantially horizontally to the support substrate 206 as shown, or the lance can be positioned in any other orientation capable of insertion into the cell 208. For a given design, the distance between the delivery pipette 204 and the lance 202 may or may not be sufficiently close to one another to cause the delivery pipette 204 to interfere with the injection procedure. In some cases, potential interference may vary depending on the size and structure of the object receiving the injection. For situations where interference may occur, it can be beneficial for the coupling between the delivery pipette and/or the lance to be adjustable to increase the distance between these components once the biological material is associated with the lance. As one non-limiting example, the delivery pipette can be pulled back from the lance tip following ejection of the biological material.

The electrostatic association and dissociation of a biological material to the lance can be further facilitated by the use of a counter or return electrode to complete an electrical circuit within the liquid buffer surrounding the lance. In some aspects the counter electrode can be positioned at any location that is in contact with the liquid buffer, including via a salt bridge or other conductive medium. In other aspects, the counter electrode can be positioned relative to the lance to influence the directionality of the flow of current in the liquid. In yet other aspects, the counter electrode can be physically coupled to the housing, thus maintaining a fixed relative position relative to the lance (unless the components of the device are adjustable). In such cases, it can be beneficial to electrically isolate the counter electrode from the lance within the housing, so that an electrical circuit is formed within the liquid during use.

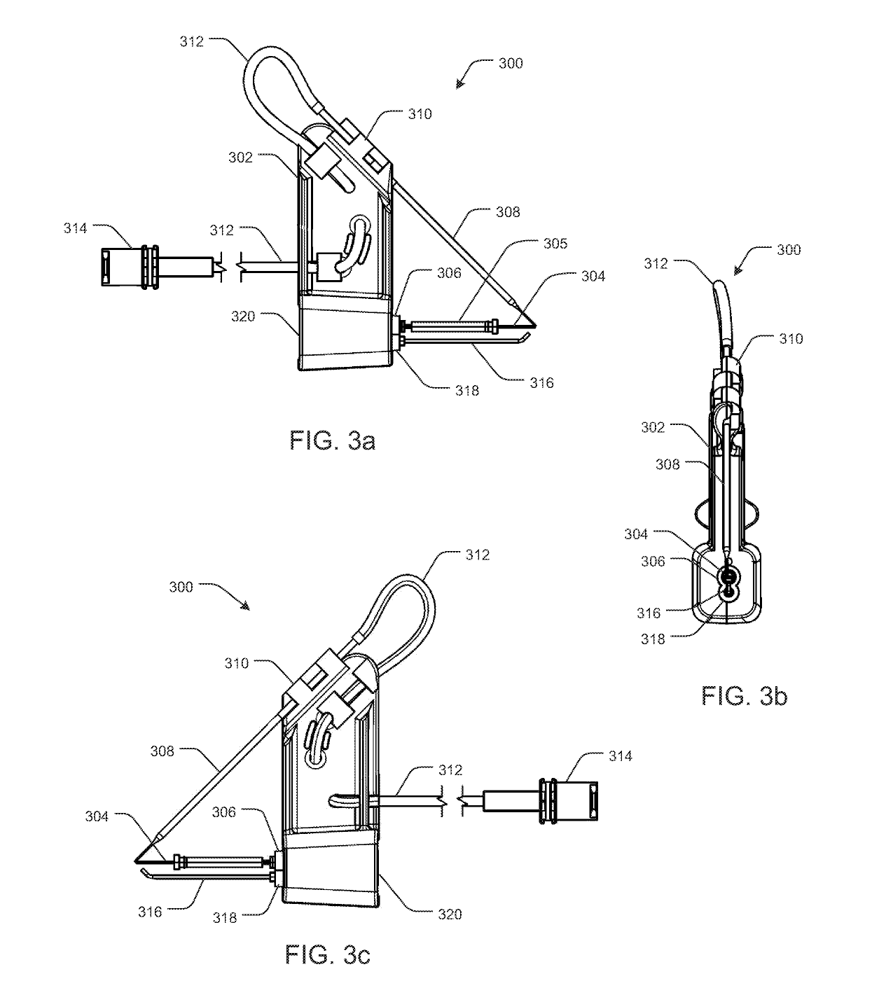

In one aspect, as is shown in FIGS. 3a-c, one design of an integrated injection device 300 is shown from the side (FIG. 3a), front (FIG. 3b), and opposite side (FIG. 3c). Such a device can include a housing 302 for holding various components in specific positions relative to one another. For example, a lance 304 is coupled to the housing 302 via a lance mount 306. A lance holder 305 can optionally be used to facilitate handling of the lance. A delivery pipette 308 is coupled to the housing 302 via a delivery pipette mount 310. The delivery pipette 308 is positioned to eject biological material contained therein onto the tip of the lance 304. Pipette tubing 312 is coupled to the delivery pipette 308 and configured to cause ejection of the biological material by fluidic pressure (e.g. liquid or gas). In one aspect, the pipette tubing 312 can be optionally looped and passed through the housing 302 as shown in FIG. 3a for strain relief purposes to protect the delivery pipette. A pipette tubing coupler 314 can be associated with the distal end of the pipette tubing 312 to provide connectivity with the micromanipulation apparatus (i.e. fluidics control). The coupler can vary depending on the design of a given micromanipulation interface. In one example, however, the coupler can be a luer fitting to facilitate the fluidic connection.

In some aspects, a counter electrode 316 can be coupled to the housing via a counter electrode mount 318 in order to complete an electrical circuit during use. The lance mount 306 and the counter electrode mount 318 can be the same or different, depending on the design of the counter electrode and the lance. The respective mounts provide mechanical support as well as electrical connectivity when the lance and counter electrode are coupled thereto. The housing 302 can additionally include a mounting interface 320 to facilitate connection between the integrated injection device 300 and various micromanipulation and/or electronic devices associated with an injection system. The mounting interface 320 can vary depending on the device to which it couples. In some aspects the mounting interface 320 can include electrical contacts to interface with and provide electrical connectively between a charging system and the lance and counter electrode mounts 306, 318. The present scope includes any physical interface design useful for connecting the housing 302 to additional system components and/or supports.

Furthermore, it is noted that while the lance, the delivery pipette, and the counter electrode are shown coupled to the housing, it is also contemplated that in some aspects each component can be mounted to a separate housing that can be further coupled to the main housing. In this manner, the components can be mixed and matched, as well as selectively replaced. Thus, for example, a lance can be coupled to the housing as shown in FIG. 3a, and a delivery pipette mounted on a separate delivery pipette housing can be removably mounted to the main housing in preparation for an injection procedure.

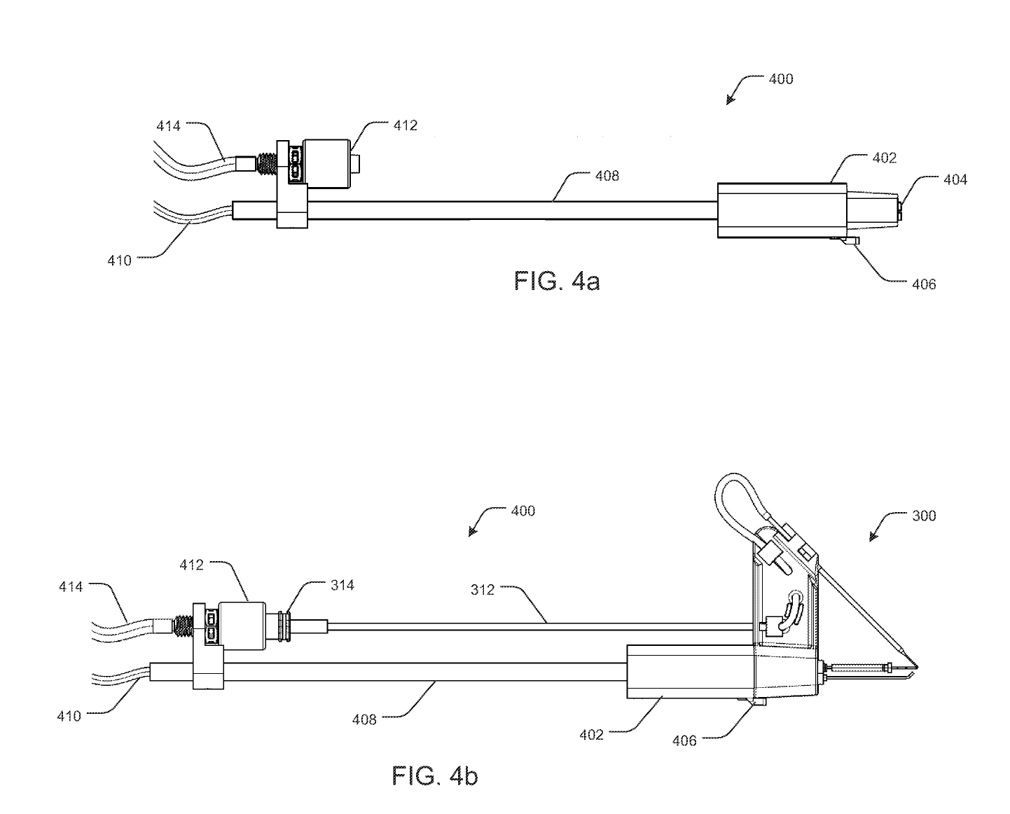

FIGS. 4a-b show exemplary aspects of a coupling device 400 for interfacing between an integrated injection device 300 and a micromanipulator and injection system (not shown). FIG. 4a shows the coupling device 400 having a mount support 402 physically configured to engage the mounting interface 320 of the integrated injection device 300 from FIGS. 3a-c. The mounting support 402 can include electrical contacts 404 configured to provide electrical connectivity with the lance and counter electrode mounts 306, 318. An alignment feature 406 can optionally be associated with the mount support 402 to assure proper engagement with the mounting interface 320. The mount support 402 can be coupled to a shaft 408 to provide connectivity with a micromanipulation device, such as, for example, a traditional micromanipulator. The shaft 408 can be sized to clamp or otherwise connect to the micromanipulator. As such, the diameter and configuration of the shaft can be designed to effectively interface with a given micromanipulator. The shaft can be solid or hollow, depending on the design of the device. As is shown in FIG. 4a, the shaft 408 is a tube though which electrical wiring 410 passes from the electrical contacts 404 to the charging system electronics.

The coupling device 400 can also include a fluidic fitting 412 configured to interface with the pipette tubing coupler 314 of the pipette tubing 312. The fluidic fitting 412 can be any type of connector capable of transmitting pressure through the tubing and that is capable of coupling with the pipette tubing coupler 314. In one non-limiting aspect, the fluidic fitting 412 can be a matching luer fitting to the pipette tubing coupler 314. Further fluidic tubing 414 can be coupled to the fluidic fitting 412 in order to provide fluidic continuity with the manipulator used to eject biological material from the delivery pipette. FIG. 4b shows an integrated injection device 300 coupled to the coupling device 400 of FIG. 4a.

It is noted that an integrated injection device and/or system can greatly enhance electrostatic injection procedures. Such an all-in-one design minimizes alignment difficulties, reduces waste of biological material, enhances biological material association with the lance, and can decrease variability between injection procedures that can arise from separate injection components.

It is noted that a variety of lance configurations and lance materials are contemplated, and any such configuration is considered to be within the present scope. In one aspect, for example, the lance can be a solid or semisolid structure, and in some cases can have an internal channel. The lance is electrically conductive and capable of holding a charge, at least at the working tip portion. Non-limiting examples of such materials can include metals, metal alloys, conductive ceramics, semiconductors, conductive polymers, metal-filled glass micropipettes, and any other suitable conductive material, including combinations thereof. Additionally, any size and/or shape of lance capable of delivering biological material into a cell is considered to be within the present scope.

Biological material can be delivered using the present system into a variety of cells. Both prokaryotic and eukaryotic cells are contemplated that can receive biological material, including cells derived from, without limitation, mammals, plants, insects, fish, birds, yeast, fungus, and the like. Additionally, cells can include somatic cells or germ line cells such as, for example, oocytes and zygotes. In one aspect, the cell can be an embryonic stem cell or a plurality of embryonic stem cells.

Additionally, in one aspect a biological material can be injected into a cellular organelle. It is now disclosed that an increased proportion of biological material can be injected into an organelle if the lance is through the organelle and out the other side. In this way, the surface area of the tapered lance is increased within the organelle. By increasing this surface area, the amount of biological material associated with this portion of the lance is increased, therefore increasing the amount that is taken into the cell. As such, when the lance is discharged, a greater proportion of biological material can remain in the organelle. Similarly, a lance penetrating shallowly into an organelle will deliver a lower proportion of biological material as compared to a lance that is inserted more deeply into the organelle. While any cellular organelle is contemplated, in one non-liming aspect the organelle can be a pronucleus. In another non-limiting aspect, the organelle is a nucleus.

Additionally, various types of biological materials are contemplated for delivery into a cell, and any type of biological material that can be delivered into a cell can be utilized in conjunction with the present restraint devices. Non-limiting examples of such biological materials can include DNA, cDNA, RNA, siRNA, tRNA, mRNA, microRNA, peptides, synthetic compounds, polymers, dyes, chemical compounds, organic molecules, inorganic molecules, hormones, and the like, including combinations thereof. In one aspect, the biological material can include DNA, cDNA, RNA, siRNA, tRNA, mRNA, microRNA, and combinations thereof. In another aspect, the biological material can include DNA and/or cDNA. Additionally, in some aspects biological material can also include one or more cells. Non-limiting examples can include embryonic stem cells, sperm, and the like.

Further exemplary details regarding cellular injectors, charging systems, movement systems, and restraining systems can be found in U.S. patent application Ser. Nos. 12/668,369, filed Sep. 2, 2010; 12/816,183; filed Jun. 15, 2010; 61/380,612, filed Sep. 7, 2010; and 61/479,777, filed on Apr. 27, 2011, each of which is incorporated herein by reference.

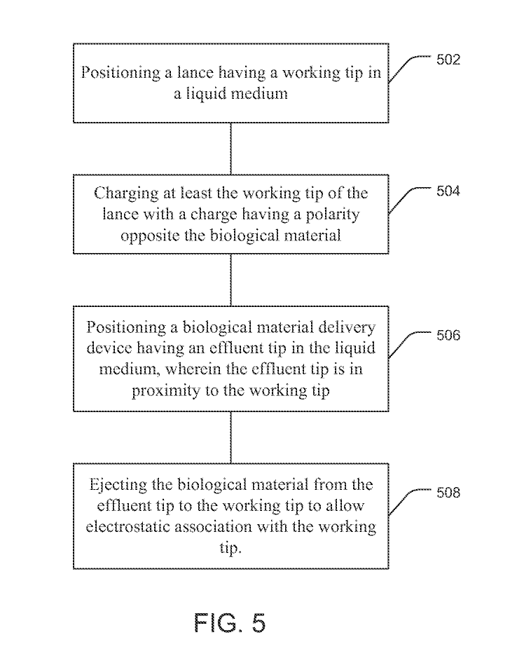

The present disclosure additionally provides methods for electrostatically associating a biological material to a lance for subsequent delivery into a cell. In one aspect, as is shown in FIG. 5 for example, such a method can include 502 positioning a lance having a working tip in a liquid medium, 504 charging at least the working tip of the lance with a charge having a polarity opposite the biological material, 506 positioning a biological material delivery device having an effluent tip in the liquid medium, wherein the effluent tip is in proximity to the working tip, and 508 ejecting the biological material from the effluent tip to the working tip to allow electrostatic association with the working tip.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed:

1. A cellular injection device, comprising:
a housing;
an injection lance coupled to the housing and having a working tip extending outward from the housing; and
a biological material delivery device coupled to the housing and having an effluent tip extending outward from the housing, the effluent tip being positioned sufficiently proximal to the working tip such that biological material expelled from the effluent tip substantially contacts the working tip.

2. The device of claim 1, wherein the injection lance is removably coupled to the housing.

3. The device of claim 1, wherein the biological material delivery device is removably coupled to the housing.

4. The device of claim 1, further comprising a counter electrode coupled to the housing and electrically isolated from the injection lance.

5. The device of claim 4, wherein the counter electrode is positioned relative to the lance to complete an electrical circuit in proximity to the working tip during use in a liquid medium.

6. The device of claim 1, wherein a distance from the effluent tip to the working tip is from about 25 microns to about 500 microns.

7. The device of claim 1, wherein a distance from the effluent tip to the working tip is from about 100 microns to about 300 microns.

8. The device of claim 1, wherein the housing further includes electrical contacts that provide electrical connection from the lance to an electrical charging device.

9. The device of claim 1, wherein the biological material delivery device further includes fluidic tubing functionally coupled thereto, wherein the fluidic tubing is coupled to the housing to provide strain relief.

10. The device of claim 1, further including a cellular injection device support removably coupled to the cellular injection device, the cellular injection device support configured to facilitate attachment of the cellular injection device to a micromanipulation device.

11. The device of claim 10, wherein the cellular injection device support provides electrical coupling between the injection lance and an electrical charging system.

12. A method of electrostatically associating a biological material to a lance for subsequent delivery into a cell, comprising:
positioning an injection lance having a working tip in a liquid medium;
charging at least the working tip of the lance with a charge having a polarity opposite the biological material;
positioning a biological material delivery device having an effluent tip in the liquid medium, wherein the effluent tip is in proximity to the working tip; and
ejecting the biological material from the effluent tip to the working tip to allow electrostatic association with the working tip.

13. The method of claim 12, wherein the working tip of the injection lance and the effluent tip of the biological material delivery device are fixed relative to one another by a housing prior to positioning in the liquid medium.

14. The method of claim 12, wherein the working tip of the injection lance and the effluent tip of the biological material delivery device are separated by a distance of from about 25 microns to about 500 microns.

15. The method of claim 12, wherein the proximity between the effluent tip and working tip is sufficient to allow the biological material to directly contact the working tip as the biological material is ejected from the effluent tip.

16. A method of injecting biological material into a cell, comprising:
introducing the device of claim 1 into a liquid medium containing a cell;
electrically charging the injection lance with a charge having a polarity opposite the biological material;
ejecting the biological material from the effluent tip to the working tip to allow electrostatic association with the working tip;
inserting the injection lance into the cell;
discharging the injection lance to release the biological material; and
withdrawing the lance from the cell.

17. The method of claim 16, further comprising coupling the device of claim 1 to a cellular injection device support, and coupling the cellular injection device support to a micromanipulation device prior to introducing the device of claim 1 into the liquid medium.

18. The method of claim 16, wherein injection lance is inserted into an organelle of the cell to inject biological material therein.

19. The method of claim 18, wherein inserting the injection lance into the organelle further includes passing a tip portion of the injection lance through the organelle to increase the surface area of the injection lance within the organelle.

20. The method of claim 19, wherein the organelle is a pronucleus.
